(19) United States
(12) Patent Application Publication
(10) Pub. No.: US 2024/0395356 A1
(43) Pub. Date: Nov. 28, 2024

(54) SYSTEMS AND METHODS FOR IDENTIFYING NOVEL PORE-FORMING TOXINS

(71) Applicants: BASF SE, Ludwigshafen am Rhein (DE); University of Southern California, Los Angeles, CA (US)

(72) Inventors: Theju Jacob, Morrisville, NC (US); Theodore Kahn, Apex, NC (US); Yan Liu, Los Angeles, CA (US); Nan Xu, Los Angeles, CA (US)

(73) Assignees: BASF SE, Florham Park, NJ (US); University of Southern California, Los Angeles, CA (US)

(21) Appl. No.: 18/558,275

(22) PCT Filed: May 5, 2022
(86) PCT No.: PCT/US2022/072132

§ 371 Date: Oct. 31, 2023

Related U.S. Application Data
(60) Provisional application No. 63/184,731, filed on May 5, 2021.
(60) Provisional application No. 63/313,134, filed on Feb. 23, 2022.

(51) Int. Cl. G16B 15/00; G06F 18/23213; G16B 30/00; G16B 40/30; G16B 45/00

(52) U.S. Cl. CPC G16B 15/00; G06F 18/23213; G16B 30/00; G16B 40/30; G16B 45/00

(57) ABSTRACT

The present disclosure relates to the field of biotechnology, and, more specifically, to systems and methods for identifying novel pore-forming toxins (PFTs) based on protein structures and sequences.

### CROSS-REFERENCE TO RELATED APPLICATIONS

This application claims the benefit of U.S. Provisional Patent Application No. 63/313,134, entitled Systems And Methods For Identifying Novel Pore-Forming Toxins, and filed on Feb. 23, 2022, and U.S. Provisional Patent Application No. 63/184,731, entitled Probabilistic Graphical Models For Pesticidal Proteins, and filed on May 5, 2021, which are expressly incorporated by reference herein in its entirety.

### FIELD OF TECHNOLOGY

The present disclosure relates to the field of biotechnology, and, more specifically, to systems and methods for identifying novel pore-forming toxins (PFTs) based on protein structures and sequences.

### BACKGROUND

Pore-forming toxins are a class of proteins that form lesions in biological membranes. Better understanding of the structure and function of PFTs will be benefit a variety of biotechnological applications. For example, bacteria that are pathogenic to insects frequently produce PFTs that target insect gut cells, and these PFTs have found widespread use in agriculture for pest control. Due to pest resistance and the need for more potent pesticides, there has been an increased interest in the search for new PFTs in recent years.

PFTs can be broadly grouped into two families, α and β pore formers, each of which is composed of proteins that use similar mechanisms to produce pores that are structurally similar. Sequence homology-based approaches such as the basic local alignment search tool (BLAST) and hidden Markov models (HMM) have been traditionally used to search for new PFTs. However, because these approaches rely only on primary sequence information for profile construction and protein searching, they fail to discover truly novel PFTs with minimal sequence commonality to known ones.

Furthermore, given that the number of known structures for PFTs is very limited, it is quite challenging to identify new PFTs having similar structures using computational approaches like deep learning, which require thousands of data samples for sufficient training. The insufficient number of training samples would lead to under-fitting, and therefore to inaccurate predictions.

### SUMMARY OF VARIOUS ASPECTS OF THE INVENTION

To address these and other needs, aspects of the present disclosure describe methods and systems for identifying novel PFTs based on protein sequence and structure data. In a first general aspect, such methods may comprise identifying, in a dataset comprising PFT information, a plurality of proteins with known sequences and structures; determining a plurality of protein clusters based on pairwise structural similarity values of the plurality of proteins; for each respective protein cluster of the plurality of protein clusters, identifying a respective group of proteins that have a pairwise sequence identity a) lower than a threshold pairwise sequence identity, b) above a threshold pairwise sequence identity, or c) within a predetermined pairwise sequence identity range; generating a graphical model trained using sequence and structure data of proteins from each respective group of proteins, wherein the graphical model is configured to generate a structural segmentation of an input protein based on a sequence of the input protein; calculating a segment interaction score for the generated structural segmentation of the input protein, wherein the segment interaction score compares the generated structural segmentation of the input protein with structures of the proteins from each respective group of proteins; and in response to determining that the segment interaction score is greater than a threshold segment interaction score, classifying the input protein as a potential novel PFT.

In some aspects, the method further comprises determining whether the sequence of the input protein is classified as a PFT sequence using a machine learning model configured to classify sequences as a PFT sequence or a non-PFT sequence; and in response to determining that the sequence is classified as a PFT sequence, identifying the input protein as a novel PFT.

In some aspects, the method further comprises receiving confirmation that the input protein is not a novel PFT; and re-training the machine learning model such that the sequence of the input protein is identified as a non-PFT sequence.

In some aspects, the method further comprises generating, for output on a computing device, an indication that the input protein is classified as a potential novel PFT and that the input protein shares a functionality of a particular protein cluster from the plurality of protein clusters.

In some aspects, determining the plurality of protein clusters further comprises: mapping a structural representation of each protein from the plurality of proteins from a high-dimensional space to a two-dimensional space that preserves structural correlations among the plurality of proteins, and executing a clustering algorithm on the two-dimensional space to determine the plurality of protein clusters.

In some aspects, the clustering algorithm is a K-means clustering algorithm.

In some aspects, generating the graphical model further comprises: aligning structures of the proteins from each respective group of proteins to identify common structural regions using an iterative pairwise alignment algorithm; and identifying consensus and non-consensus secondary structure segments in the aligned structures, wherein training the graphical model comprises maximizing a probability of the identified consensus and non-consensus secondary structure segments in the structural segmentation of the input protein.

In some aspects, the graphical model is a semi-Markov conditional fields (semi-CRFs) model.

In some aspects, proteins in a respective group of proteins share functionality and have a low sequence identity, optionally less than 80, 70, 60, 50, 40, 30, 20, or 10% full length sequence identity.

In a second general aspect, the disclosure provides computer-implemented systems comprising at least one processor configured to execute instructions for carrying out any of the methods described herein, or any subset of step(s) thereof.

It should be noted that the aspects described herein may be implemented in a system comprising a hardware processor. Alternatively, such methods may be implemented using computer-executable instructions stored in a non-transitory computer readable medium.

The above simplified summary of exemplary aspects serves to provide a basic understanding of the present disclosure. This summary is not an extensive overview of all contemplated aspects, and is not intended to identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more exemplary aspects of the present disclosure and, together with the detailed description, serve to explain their principles and implementations.

FIG. 1 illustrates a diagram of a system for identifying novel PFTs based on protein structures and sequences, in accordance with aspects of the present disclosure.

FIG. 2 is a diagram of clusters formed based on structural representations of proteins mapped in a two-dimensional space, in accordance with aspects of the present disclosure.

FIG. 3 is a block diagram illustrating an example protein structure graph constructed with consensus secondary structures, in accordance with aspects of the present disclosure.

FIG. 4 illustrates a flow diagram of an exemplary method for identifying novel PFTs based on protein structures and sequences, in accordance with aspects of the present disclosure.

FIG. 5 illustrates an example of a general-purpose computer system on which aspects of the present disclosure can be implemented.

### DETAILED DESCRIPTION

Exemplary aspects are described herein in the context of a system, method, and computer program product for identifying novel PFTs based on protein structures and sequences. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the exemplary aspects as illustrated in the accompanying drawings. The same reference indicators will be used to the extent possible throughout the drawings and the following description to refer to the same or like items.

The functions of PFTs correlate closely with their structures. Accordingly, a search methodology utilizing structures overcomes the before-mentioned limitations of sequence-based approaches. The present disclosure presents such a search methodology, and in some aspects uses a sample-efficient graphical model, in which a protein structure graph is first constructed according to consensus secondary structures. A Semi-Markov Conditional Random Fields model is then developed to perform protein sequence segmentation. For this approach, both sequence and structure data are fully utilized to learn intra- and inter-segment interactions during model training. For a protein being tested using this model, only the sequence information is required to determine how likely it is to have a structure similar to that of the PFTs from the training set.

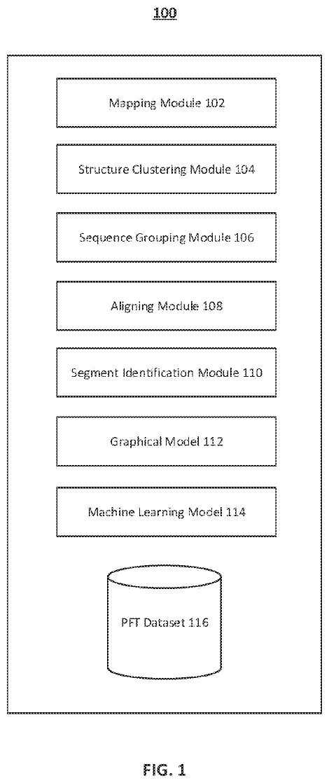

FIG. 1 illustrates a diagram of system 100 for identifying novel PFTs based on protein structures and sequences, in accordance with aspects of the present disclosure. System 100 includes a plurality of modules, namely: mapping module 102, structure clustering module 104, sequence grouping module 106, aligning module 108, segment identification module 110, graphical model 112, machine learning model 114, and PFT dataset 116. Each module in system 100 may be configured to perform a specific task and will be described further below.

The first step in identifying new PFTs involves generating a dataset (e.g., PFT dataset 116) that can be used to train graphical model 112. PFT dataset 116 includes information about PFTs with known sequence and structure data. In some aspects, PFT dataset 116 encompasses a plurality of pore-forming protein families (e.g., 16 families) that are functionally and evolutionarily related. From these families, a plurality of proteins may be extracted (e.g., N=171 unique proteins).

Dataset construction further comprises identifying, using structure clustering module 104, clusters of proteins with similar functions according to their pairwise structural similarities. A protein i can be represented with vector mᵢ∈R^{+N}, where m_{i,j} indicates structural similarity with protein j and N is total number of proteins in the plurality of proteins. To identify groups of proteins sharing similar structures automatically, mapping module 102 maps the structure representation for proteins in high-dimensional space to a 2D space using a visualization technique (e.g., t-SNE), which helps both preserve and visualize structural correlations among proteins. Structure clustering module 104 may then execute a clustering algorithm (e.g., using a K-means clustering algorithm) on the mapped structure representations.

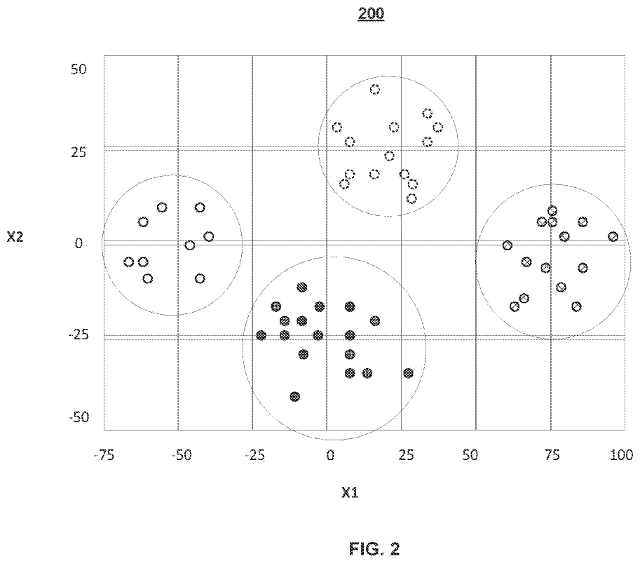

FIG. 2 is diagram 200 of clusters formed based on structural representations of proteins mapped in a 2D space, in accordance with aspects of the present disclosure. Proteins within each cluster have much higher structural similarity compared with those in other clusters. It should be noted that structurally-similar proteins share similar intramolecular distances between equivalent pairs of atoms. On the other hand, proteins are considered to be sequentially similar when their amino acid sequences display considerable overlap. In FIG. 2, there are four clusters of proteins identified in the 2D space. The axes represent principal components of the underlying data elements.

In an exemplary aspect, sequence grouping module 106 may then identify twilight zone protein clusters. The twilight zone refers to one or more groups of proteins within a protein cluster that have a pairwise sequence identity that is lower than a threshold pairwise sequence identity (e.g., 0.4). Proteins in this cluster represent proteins with high structural similarity and minimal sequence commonality (because of the low sequence identity). For example, groups of proteins with similar structures but low sequence identity are shown in table 1 below:

TABLE 1
Groups of proteins

| Groups | I (9 proteins) | II (4 proteins) | III (9 proteins) |
|---|---|---|---|
| Proteins | Endotoxin_C: | Clenterotox: | MACPF: |
| | 1DLCA, 1I5PA, | 2XH6A | 2QP2A, 2QQHA |
| | 1W99A, 2C9KA, | 2ZOEA | 3NSJA, 3OJYB |
| | 4ARXA, 4DBMA, | 3WINE | 5J67A, 5OUPA |
| | 5ZI1A, 60WKA, | | 6CXOA, 4OEJA |
| | Thiol_cytolysin: | BB_PF: | Thiol_cytolysin: |
| | 4ZGHA | 4MJTA | 2BK2A |

In table 1, each group has a pairwise sequence identity that is less than a threshold pairwise sequence identity. For example, group I represents proteins in a twilight zone from a first cluster, group II represents proteins in a twilight zone from a second cluster, and group III represents proteins in a twilight zone from a third cluster. Given sequence and structural information of proteins in the twilight zones, novel PFTs can thus be discovered that share similar functionality, even though their sequence similarity to known PFTs is low.

More specifically, for a group of PFTs with high structural similarity but low sequence identity, a graphical model can be trained with their sequence and structure data. The graphical model learns the underlying shared patterns and can be further utilized to discover additional PFTs with similar function, without knowing their three-dimensional structures in advance.

In terms of generating graphical model 112, first, for proteins with high structural similarity (e.g., proteins within a twilight zone), aligning module 108 performs structural alignment by executing alignment algorithms such as POSA, which is described in Li Z, et al. POSA: a user-driven, interactive multiple protein structure alignment server. Nucleic Acids Research, 2014. Multi-protein structural alignment is an important approach for functional and evolutionary analysis of groups of protein structures. In the present disclosure, structures from multiple proteins are aligned to identify conserved regions, which form the common structural core in the targeted twilight zones. Although differing in both the heuristics used to find the approximate solution and the type of scoring function used, the majority of existing multiple protein structure alignment algorithms (e.g., STAMP, MALECON, etc.) use the same tabular row-column representation, leading to many limitations. Essentially, the tabular row-column representation provides very limited information about similarities present only in a subset of proteins being aligned, which results in a very small conserved protein core in most multiple structure comparisons in the end.

Instead, an algorithm such as POSA with a partial-order graph representation for multiple alignments is adopted by aligning module 108. Specifically, the multiple protein structure alignment is formulated by a process of iterative pairwise alignment of two multiple structure alignments, each represented as a directed acyclic graph. In addition, constraints of consecutiveness must be obeyed in aligning two partial-order graphs (POGs), which means two residues that have no order relationship or have wrong order can never be found in an alignment.

Of particular interest are protein secondary structure elements, which are local folded structural elements within the protein structure and may fall into two categories: α helices or β sheets. Secondary structures in a protein are regions stabilized by hydrogen bonds between atoms in the polypeptide backbone. In terms of alignment, proteins from group II in table 1, for example, may have a certain number (e.g., 5) of segments of consensus secondary structures that align well with each other. For example, consensus secondary structure may be determined based upon an 8-state secondary structure prediction algorithm.

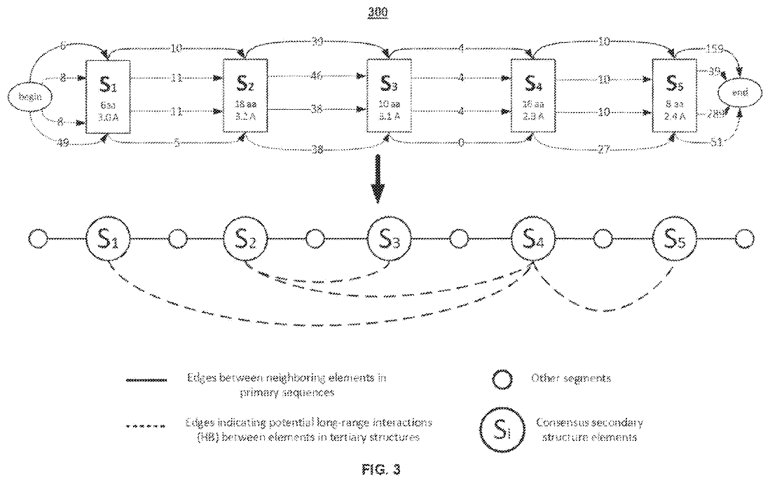

FIG. 3 is a block diagram illustrating an examplary protein structure graph 300 constructed with consensus secondary structures, in accordance with aspects of the present disclosure. With proteins from each group segmented by consensus and non-consensus secondary structures after structure alignment, protein structure graphs with elements as shown in FIG. 3 are constructed. For example, in FIG. 3, protein structure graph 300 is composed of consensus secondary structure segments S₁, S₂, S₃, S₄, and S₅, and six other segments connected in a primary sequence.

Depending on the structure alignment, segment identification module 110 connects consensus segments to each other either with or without the partition of other segments. Considering that toxins are either α or β pore-formers with hydrogen bonds in their secondary structures, the 3D structure visualization for each protein is reviewed by segment identification module 110 and hydrogen-bonded consensus secondary structure segments in the protein structure graph are connected. In FIG. 3, edges between S₁ and S₄, S₂ and S₃, S₂ and S₄, and S₄ and S₅ indicate potential long-range interactions between elements in tertiary structures.

In an exemplary aspect, the protein structure with secondary structures of the training proteins are given as inputs to the graphical model 112. For example, graphical model 112 (e.g., a semi-Markov conditional random fields (semi-CRFs) model) may be employed to maximize the probability of observed consensus and non-consensus secondary structure segments in the protein structure graph. The j-th segment from the protein sequence is denoted as:

$${s}_{j} = < {t}_{j} , {u}_{j} , {y}_{j} > ,$$

where tⱼ and uⱼ are the start and end positions, while yⱼ is a label (0 for all non-consensus segments and segment index for consensus segments). If K features are considered for each segment and the k-th feature function for the j-th segment from sequence s is g^{k} (j, x, s)∈R, then the segment feature functions are represented by g=<g^{j}, . . . , g^{K}> and the global feature function for sequence x with segmentation s becomes.

$$G ( x , s ) = {\sum }_{j}^{\vert s \vert} g ( j , x , s )$$

Thus, the conditional probability of segmentation s for protein sequence x with model parameter W is:

$$P ( s \vert x , W ) = \frac{1}{Z ( x )} {e}^{W \cdot G ( x , s )} ,$$

where Z(x) is the normalization factor with value Σₛ·e^{W·G(x,s′)}. In some aspects, this probability is optimized by the stochastic gradient algorithm. For example, the gradient-based training method SGA-ADADELTA with L2 regularization may be adopted to learn parameters of the constructed semi-CRFs model. Specifically, the following hyperparameters are used to learn model parameters. For number of epochs, approximately 10 epochs may be used to go through the sequences. In terms of tolerance, to avoid overfitting to the small dataset, an early stopping policy with a pre-defined threshold 1e-6 may be adopted (i.e., training ends once the relative difference between the estimated average log-likelihood across all sequences between the current and previous epoch is below 1e-6). In some aspects, another strategy to mitigate the risk of overfitting is to take advantage of L2 regularization with a coefficient of 1.

For a new test protein with only sequence information known, the segmentation with well-trained graphical model 112 is first inferred, and then a segment interaction score is calculated to reflect how likely the new protein is to have a similar structure to the training proteins. The amino acid pairing preference is computed as m_{i,j}=P(Aᵢ: Aⱼ)/P(Aᵢ)P(Aⱼ) for residual-level interaction. For two segments x−X₁ . . . Xₘ and y−Y₁ . . . Yₙ, the segment-level alignment score is:

$$score ( x , y ) = {\sum }_{i} {\sum }_{j} ( {m}_{i , j} I ( parallel β - strand ) + {{m}^{′}}_{i , j} I ( anti - parallel β - strand ) ,$$

Where I is an indicator function suggesting whether the two segments are anti-parallel beta-strand. For two anti-parallel beta-strands s and s′, the anti-parallel alignment score is computed by: f_{PAS} (s, s′)=score (x,y); otherwise, f_{PAS}(s, s′)=0. Finally, the segmentation interaction score is computed over all pairs of segments:

$$score ( S ) = {\sum }_{{s}_{i} , {s}_{j} ϵ s} {f}_{PAS ( {s}_{i} , {s}_{j} )}$$

The greater the segmentation interaction score, the more structurally similar the test protein is to the training proteins, and the more likely it is to share the functionality of that PFT group. In response to determining that the segmentation interaction score is greater than a threshold segmentation interaction score, system 100 may determine that an input protein of graphical model 112 is a potential novel PFT. In some aspects, for graphical model training and inference, both intra-segment features (e.g., amino acid type, Atchley factor, 3- and 8-state secondary structure predictions, etc.) and inter-segment features (e.g., parallel β-sheet alignment score) are utilized.

As mentioned previously, potential PFTs may be filtered with both sequence and structure-based models. Given the large numbers (8K˜400K) of proteins with potentially similar structural patterns as positive proteins in three twilight zones, in some aspects, the proposed graphical model may be applied alongside machine learning model 114 such as a sequence-based deep neural network (e.g., ProtCNN) to select proteins with high probabilities of having similar functionalities as the three studied groups of PFTs.

For a new testing protein with only sequence information known, the segmentation may be inferred with a trained graphical model 112 first, and then the segmentation interaction-based score may be calculated to estimate how likely it is to have a similar structure to the training proteins. From this perspective, the threshold is set as the ranking score of the positive-testing protein (e.g., a protein with known structure that is confirmed as a PFT) and will only keep testing proteins with higher ranking scores.

In addition, machine learning model 114 (e.g., a convolutional neural network such as ProtCNN) may predict which protein family the testing protein comes from. In some aspects, the convolutional neural network may be adapted by adjusting the network architecture to make binary decisions (i.e., whether the protein is a pore-forming toxin or not). The training dataset for the convolutional neural network may include a plurality of sequences of which some are PFTs and the rest are not. Ultimately, proteins with the structural ranking score higher than the testing positive protein in each group and a high probability of being PFTs estimated by the convolutional neural network are considered into the final candidate list to be evaluated in lab experiments.

In some aspects, subsequent to the convolutional neural network indicating whether a new protein is a PFT, the new protein can be tested in a laboratory experiment to confirm whether the new protein actually behaves like a PFT. If the new protein is not a PFT, the training dataset can be updated to include an entry with the sequence of the new protein correctly classified as a non-PFT.

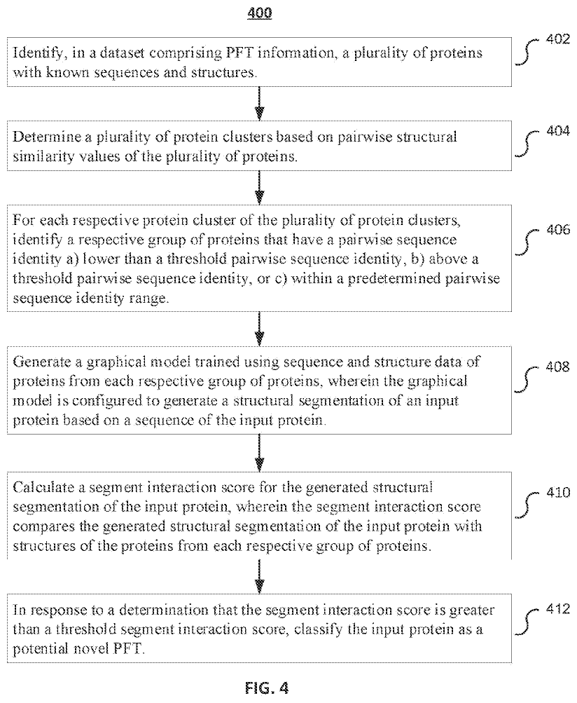

FIG. 4 illustrates a flow diagram of an exemplary method 400 for identifying novel PFTs based on protein structures and sequences, in accordance with aspects of the present disclosure.

At 402, method 400 comprises a step of identifying, in a dataset comprising PFT information, a plurality of proteins with known sequences and structures. At step 404, a plurality of protein clusters is determined based on pairwise structural similarity values of the plurality of proteins. At step 406, for each respective protein cluster of the plurality of protein clusters, a respective group of proteins is identified which has a pairwise sequence identity in accordance with a desired parameter, e.g., a) lower than a threshold pairwise sequence identity, b) above a threshold pairwise sequence identity, or c) within a predetermined pairwise sequence identity range, as shown by this figure. In other aspects, the one or more respective groups of proteins may be identified based on other criteria (secondary structure, motifs, etc.). A step 408, a graphical model trained using sequence and structure data of proteins from each respective group of proteins may be generated, wherein the graphical model is configured to generate a structural segmentation of an input protein based on a sequence of the input protein. A segment interaction score may then be calculated at step 410 for the generated structural segmentation of the input protein, wherein the segment interaction score compares the generated structural segmentation of the input protein with structures of the proteins from each respective group of proteins. Finally at step 412, in response to determining that the segment interaction score is greater than a threshold segment interaction score, the input protein may be classified as a potential novel PFT. One or more of the foregoing steps may be performed using a computer-implemented system as described herein.

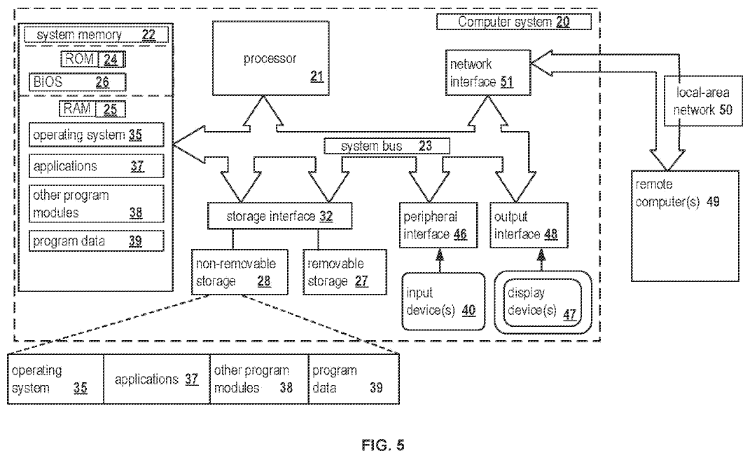

FIG. 5 is a block diagram illustrating a computer system 20 on which aspects of systems and methods for identifying novel PFTs based on protein structures and sequences may be implemented in accordance with an exemplary aspect. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a graphics processing unit (GPU), a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I²C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-4 may be performed by processor 21 (e.g., processor 21 may execute the components of system 100). The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM. SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It will be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A method for identifying novel pore-forming toxins (PFTs) based on sequence and structure data, comprising:
identifying, in a dataset comprising PFT information, a plurality of proteins with known sequences and structures;
determining a plurality of protein clusters based on pairwise structural similarity values of the plurality of proteins;
for each respective protein cluster of the plurality of protein clusters, identifying a respective group of proteins that have a pairwise sequence identity a) lower than a threshold pairwise sequence identity, b) above a threshold pairwise sequence identity, or c) within a predetermined pairwise sequence identity range;
generating a graphical model trained using sequence and structure data of proteins from each respective group of proteins, wherein the graphical model is configured to generate a structural segmentation of an input protein based on a sequence of the input protein;
calculating a segment interaction score for the generated structural segmentation of the input protein, wherein the segment interaction score compares the generated structural segmentation of the input protein with structures of the proteins from each respective group of proteins; and
in response to determining that the segment interaction score is greater than a threshold segment interaction score, classifying the input protein as a potential novel PFT.

2. The method of claim 1, further comprising:
determining whether the sequence of the input protein is classified as a PFT sequence using a machine learning model configured to classify sequences as a PFT sequence or a non-PFT sequence; and
in response to determining that the sequence is classified as a PFT sequence, identifying the input protein as a novel PFT.

3. The method of claim 2, further comprising:
receiving confirmation that the input protein is not a novel PFT; and
re-training the machine learning model such that the sequence of the input protein is identified as a non-PFT sequence.

4. The method of claim 1, further comprising generating, for output on a computing device, an indication that the input protein is classified as a potential novel PFT and that the input protein shares a functionality of a particular protein cluster from the plurality of protein clusters.

5. The method of claim 1, wherein determining the plurality of protein clusters further comprises:
mapping a structural representation of each protein from the plurality of proteins from a high-dimensional space to a two-dimensional space that preserves structural correlations among the plurality of proteins; and
executing a clustering algorithm on the two-dimensional space to determine the plurality of protein clusters.

6. The method of claim 5, wherein the clustering algorithm is a K-means clustering algorithm.

7. The method of claim 1, wherein generating the graphical model further comprises:
aligning structures of the proteins from each respective group of proteins to identify common structural regions using an iterative pairwise alignment algorithm; and
identifying consensus and non-consensus secondary structure segments in the aligned structures, wherein training the graphical model comprises maximizing a probability of the identified consensus and non-consensus secondary structure segments in the structural segmentation of the input protein.

8. The method of claim 1, wherein the graphical model is a semi-Markov conditional fields (semi-CRFs) model.

9. The method of claim 1, wherein proteins in a respective group of proteins share functionality and have a low sequence identity, optionally less than 60, 50, 40, 30, 20, or 10% full length sequence identity.

10. A system for identifying novel pore-forming toxins (PFTs) based on sequence and structure data, comprising:
a processor, and
memory having stored therein instructions that when executed by the processor cause the processor to:
identify, in a dataset comprising PFT information, a plurality of proteins with known sequences and structures;
determine a plurality of protein clusters based on pairwise structural similarity values of the plurality of proteins;
for each respective protein cluster of the plurality of protein clusters, identify a respective group of proteins that have a pairwise sequence identity a) lower than a threshold pairwise sequence identity, b) above a threshold pairwise sequence identity, or c) within a predetermined pairwise sequence identity range;
generate a graphical model trained using sequence and structure data of proteins from each respective group of proteins, wherein the graphical model is configured to generate a structural segmentation of an input protein based on a sequence of the input protein;
calculate a segment interaction score for the generated structural segmentation of the input protein, wherein the segment interaction score compares the generated structural segmentation of the input protein with structures of the proteins from each respective group of proteins; and
in response to determining that the segment interaction score is greater than a threshold segment interaction score, classify the input protein as a potential novel PFT.

11. The system of claim 10, wherein the memory further comprises instructions for:
determining whether the sequence of the input protein is classified as a PFT sequence using a machine learning model configured to classify sequences as a PFT sequence or a non-PFT sequence; and
in response to determining that the sequence is classified as a PFT sequence, identifying the input protein as a novel PFT.

12. The system of claim 11, wherein the memory further comprises instructions for:
receiving confirmation that the input protein is not a novel PFT; and
re-training the machine learning model such that the sequence of the input protein is identified as a non-PFT sequence.

13. The system of claim 10, wherein the memory further comprises instructions for:
generating, for output on a computing device, an indication that the input protein is classified as a potential novel PFT and that the input protein shares a functionality of a particular protein cluster from the plurality of protein clusters.

14. The system of claim 10, wherein determining the plurality of protein clusters further comprises:
mapping a structural representation of each protein from the plurality of proteins from a high-dimensional space to a two-dimensional space that preserves structural correlations among the plurality of proteins; and
executing a clustering algorithm on the two-dimensional space to determine the plurality of protein clusters.

15. The system of claim 14, wherein the clustering algorithm is a K-means clustering algorithm.

16. The system of claim 10, wherein generating the graphical model further comprises:
aligning structures of the proteins from each respective group of proteins to identify common structural regions using an iterative pairwise alignment algorithm; and
identifying consensus and non-consensus secondary structure segments in the aligned structures, wherein training the graphical model comprises maximizing a probability of the identified consensus and non-consensus secondary structure segments in the structural segmentation of the input protein.

17. The system of claim 10, wherein the graphical model is a semi-Markov conditional fields (semi-CRFs) model.

18. The system of claim 10, wherein proteins in a respective group of proteins share functionality and have low sequence identity.
